(19) United States
(12) Patent Application Publication
(10) Pub. No.: US 2015/0037254 A1
(43) Pub. Date: Feb. 5, 2015

(54) CONTRAST AGENT FOR PHOTOACOUSTIC IMAGING

(71) Applicant: Canon Kabushiki Kaisha, Tokyo (JP)

(72) Inventors: Tatsuki Fukui, Yokohama-shi (JP); Mayuko Kishi, Machida-shi (JP); Fumiko Tomatsu, Yokohama-shi (JP); Daisuke Sasaguri, Yokohama-shi (JP); Satoshi Yuasa, Yokohama-shi (JP)

(21) Appl. No.: 14/332,641

(22) Filed: Jul. 16, 2014

(30) Foreign Application Priority Data
Jul. 30, 2013 (JP) 2013-158053

(51) Int. Cl. A61K 49/22

(52) U.S. Cl. CPC A61K 49/22
USPC 424/9.1

(57) ABSTRACT

Provided is a particle having a large photoacoustic signal per unit dye, the particle including: one of a hydrophobic metal naphthalocyanine dye and a hydrophobic metal phthalocyanine dye; one of an oil, a fatty acid, and a fatty acid ester as a matrix material; and a surfactant, in which a ratio of the weight of the matrix material to the weight of the particle is 50% or more.

### BACKGROUND OF THE INVENTION

1. Field of the Invention

The present invention relates to a contrast agent for photoacoustic imaging using a particle including a hydrophobic metal naphthalocyanine dye or a hydrophobic metal phthalocyanine dye, an oil, a fatty acid, or a fatty acid ester, and a surfactant.

2. Description of the Related Art

In recent years, a photoacoustic imaging method has been attracting attention as an imaging method that enables a noninvasive diagnosis.

The photoacoustic imaging method is a method involving detecting the strength of an acoustic wave generated by volume expansion caused by heat emitted from a molecule as a measuring object irradiated with light and the position at which the acoustic wave is generated to provide an image of the measuring object. In the photoacoustic imaging method, a dye can be used as a contrast agent for increasing the magnitude of fluorescence from a site to be measured or the strength of the acoustic wave.

Here, Investigative Ophthalmology & Visual Science, November 1995, Vol. 36, No. 12 discloses an example in which a silicon naphthalocyanine derivative (hereinafter sometimes abbreviated as “SiNc”) as a dye is solubilized by dissolving the dye in canola oil and mixing the solution with a surfactant. An animal experiment has been performed with an eye toward its application to a photodynamic therapy.

Meanwhile, Japanese Patent Application Laid-Open No. 2005-246013 discloses an example in which a particle containing a matrix material such as an antioxidant vitamin is produced by using a phthalocyanine dye as a dye and the particle is used in a photochemical therapy.

However, the solubilized product containing the SiNc disclosed in Investigative Ophthalmology & Visual Science, November 1995, Vol. 36, No. 12 is merely such that the dye is solubilized so as to be capable of being administered to an animal, and the solubilized product involves a problem in that a photoacoustic signal per unit dye is small when the solubilized product is used in the photoacoustic imaging method. In addition, its particle diameter is not controlled and hence the solubilized product is unsuitable for use in the photoacoustic imaging method.

In addition, the matrix material for the particle containing the phthalocyanine dye disclosed in Japanese Patent Application Laid-Open No. 2005-246013 is selected from the viewpoint of its utilization in the photochemical therapy, and hence the particle involves a problem in that a photoacoustic signal per unit dye is small when the particle is used in the photoacoustic imaging method.

### SUMMARY OF THE INVENTION

In view of the foregoing, an object of the present invention is to provide a particle in which a matrix material capable of amplifying a photoacoustic signal is selected to enlarge a photoacoustic signal per unit dye.

The present invention relates to a particle including: one of a hydrophobic metal naphthalocyanine dye and a hydrophobic metal phthalocyanine dye; one of an oil, a fatty acid, and a fatty acid ester as a matrix material; and a surfactant, in which a ratio of the weight of the matrix material to the weight of the particle is 50% or more.

Further features of the present invention will become apparent from the following description of exemplary embodiments with reference to the attached drawings.

### BRIEF DESCRIPTION OF THE DRAWINGS

FIG. 1 is a view for illustrating a particle according to Embodiment 1 of the present invention.

FIG. 2 is a graph for showing the results of Examples A1 and A2, and Comparative Example B1 of the present invention.

FIG. 3 is a graph for showing the results of Example C1 and Comparative Example D1 of the present invention.

FIG. 4 is a graph for showing the results of Examples E1 and E2, and Comparative Examples F1 and F2 of the present invention.

FIG. 5 is a graph for showing the results of Examples G1, G2, and G3, and Comparative Example H1 of the present invention.

FIG. 6 is a graph for showing the results of Examples J1, J2, and J3, and Comparative Example K1 of the present invention.

### DESCRIPTION OF THE EMBODIMENTS

Preferred embodiments of the present invention will now be described in detail in accordance with the accompanying drawings.

Hereinafter, embodiments of the present invention are described but the present invention is not limited thereto.

(Construction of Embodiment)

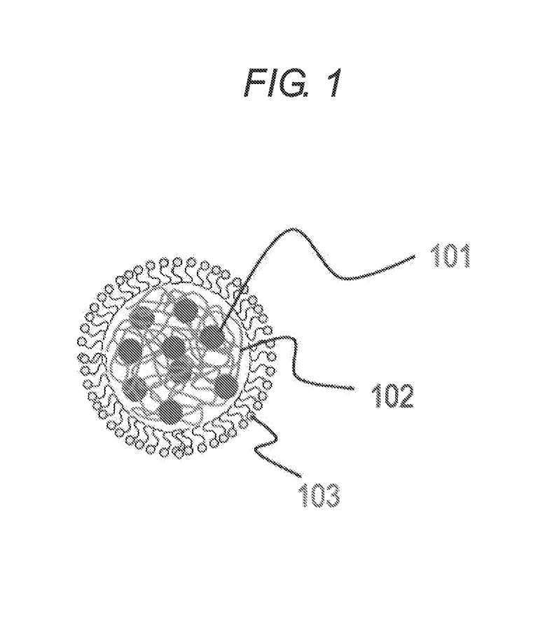

As illustrated in FIG. 1, a particle according to this embodiment is a particle including a hydrophobic metal naphthalocyanine dye or hydrophobic metal phthalocyanine dye 101, a matrix material 102, and a surfactant 103 on its surface. In addition, the hydrophobic metal naphthalocyanine dye or the hydrophobic metal phthalocyanine dye is represented by the chemical formula (1) or (2). In addition, the particle according to this embodiment has a feature in that the ratio of the weight of the matrix material to the weight of the particle is 50% or more. The hydrophobic metal naphthalocyanine dye or hydrophobic metal phthalocyanine dye according to this embodiment hardly leaks to the outside of the particle even in an aqueous solution such as serum because the dye has a structure having high hydrophobicity and is free of a hydrophilic functional group.

The reason why the strength of a photoacoustic signal increases when the particle includes the matrix material is described.

First, it has been known that the strength of the photoacoustic signal is proportional to a coefficient of thermal expansion and is inversely proportional to a specific heat capacity. In addition, the matrix material such as an oil, a fatty acid, or a fatty acid ester generally has a higher coefficient of thermal expansion and a lower specific heat capacity than those of water. Accordingly, a state where the matrix material is present in the particle and the matrix material is present around the dye has a higher strength of the photoacoustic signal than that of a state where the matrix material is absent and water is present around the dye. It should be noted that the coefficient of thermal expansion of water is 0.2 [10⁻³/° C.] and the specific heat capacity of water is 4,182 [J/kg° C.].

(Hydrophobic Metal Naphthalocyanine Dye or Hydrophobic Metal Phthalocyanine Dye)

The dye in the present invention is defined as a compound capable of absorbing light having a wavelength in the range of from 600 nm to 1,300 nm.

In addition, the hydrophobic dye in this embodiment is defined as a dye having an Rf value calculated by a thin-layer liquid chromatography (hereinafter sometimes abbreviated as “TLC”) method to be described later in Examples of 0 or more and 0.50 or less.

In this embodiment, the structure of the hydrophobic metal naphthalocyanine dye or the hydrophobic metal phthalocyanine dye is represented by the following chemical formula (1) or (2).

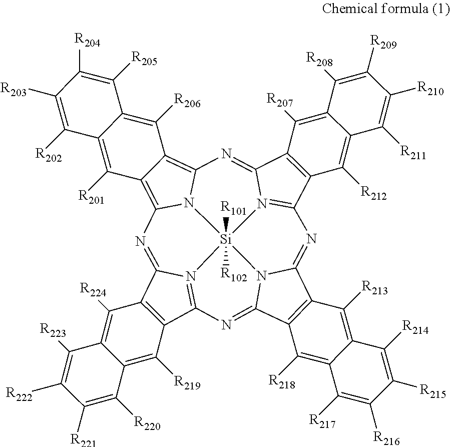

(In the formula, R₂₀₁ to R₂₂₄ may be identical to or different from one another, and each represent a hydrogen atom, a halogen atom, an acetoxy group, an amino group, a nitro group, a cyano group, or an alkyl group having 1 to 18 carbon atoms or an aromatic group that is unsubstituted or substituted with at least one functional group selected from a halogen atom, an acetoxy group, an amino group, a nitro group, a cyano group, and an alkyl group having 1 to 18 carbon atoms. In addition, R₁₀₁ and R₁₀₂ may be identical to or different from each other, and each represent —OH, —OR₁₁, —OCOR₁₂, —OSi(—R₁₃)(—R₁₄)(—R₁₅), a halogen atom, an acetoxy group, an amino group, a nitro group, a cyano group, or an alkyl group having 1 to 18 carbon atoms or an aromatic group that is unsubstituted or substituted with at least one functional group selected from a halogen atom, an acetoxy group, an amino group, a nitro group, a cyano group, and an alkyl group having 1 to 18 carbon atoms. Here, R₁₁ to R₁₅ may be identical to or different from one another, and each represent a group that is unsubstituted or substituted with at least one functional group selected from a halogen atom, an acetoxy group, an amino group, a nitro group, a cyano group, and an alkyl group having 1 to 18 carbon atoms.)

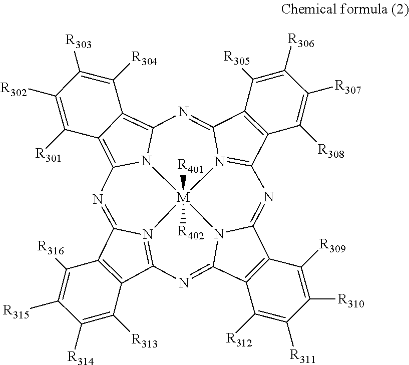

(In the formula, R₃₀₁ to R₃₁₆ may be identical to or different from one another, and each represent a hydrogen atom, a halogen atom, an acetoxy group, an amino group, a nitro group, a cyano group, or an alkyl group having 1 to 18 carbon atoms or an aromatic group that is unsubstituted or substituted with at least one functional group selected from a halogen atom, an acetoxy group, an amino group, a nitro group, a cyano group, and an alkyl group having 1 to 18 carbon atoms. In the formula, M represents one of elements Zn, Cu, Co, and Si. R₄₀₁ and R₄₀₂ may be absent depending on the element represented by M, or may each represent a structure described below. R₄₀₁ and R₄₀₂ may be identical to or different from each other, and each represent —OH, —OR₅₀₁, —OCOR₅₀₂, —OSi(—R₅₀₃)(—R₅₀₄)(—R₅₀₅), a halogen atom, an acetoxy group, an amino group, a nitro group, a cyano group, or an alkyl group having 1 to 18 carbon atoms or an aromatic group that is unsubstituted or substituted with at least one functional group selected from a halogen atom, an acetoxy group, an amino group, a nitro group, a cyano group, and an alkyl group having 1 to 18 carbon atoms. Here, R₅₀₁ to R₅₀₅ may be identical to or different from one another, and each represent a group that is unsubstituted or substituted with at least one functional group selected from a halogen atom, an acetoxy group, an amino group, a nitro group, a cyano group, and an alkyl group having 1 to 18 carbon atoms.)

The hydrophobic metal naphthalocyanine dye or hydrophobic metal phthalocyanine dye according to this embodiment has a conjugated double bond, and hence can absorb light having a specific wavelength and can be used in photoacoustic imaging.

In addition, the hydrophobic metal naphthalocyanine dye or hydrophobic metal phthalocyanine dye according to this embodiment preferably has a molar absorption coefficient of 10⁶ M⁻¹cm⁻¹ or more at at least one wavelength selected from the range of from 600 nm to 1,300 nm.

Examples of the hydrophobic metal naphthalocyanine dye can include silicon 2,3-naphthalocyanine bis(trihexylsilyloxide), silicon 2,3-naphthalocyanine dihydroxide, silicon 2,3-naphthalocyanine dioctyloxide, silicon 2,3-naphthalocyanine dichloride, and bis(di-isobutyl octadecylsiloxy) silicon 2,3-naphthalocyanine.

Examples of the hydrophobic metal phthalocyanine dye can include zinc 2,9,16,23-tetra-tert-butyl-29H,31H-phthalocyanine, copper(II) 2,9,16,23-tetra-tert-butyl-29H,31H-phthalocyanine, cobalt 2,9,16,23-tetra-tert-butyl-29H,31H-phthalocyanine, and tert-butyl silicon-bis(trimethylsiloxy)-phthalocyanine.

(Surfactant)

The particle according to this embodiment includes the surfactant. The surfactant in this embodiment is not particularly limited and any surfactant may be used as long as the surfactant can form the particle. For example, a nonionic surfactant, an anionic surfactant, a cationic surfactant, a polymeric surfactant, or a phospholipid can be used. Only one kind of those surfactants may be used, or two or more kinds thereof may be used.

Examples of the nonionic surfactant can include: polyoxyethylene sorbitan-based fatty acid esters such as Tween (trademark) 20, Tween (trademark) 40, Tween (trademark) 60, Tween (trademark) 80, and Tween (trademark) 85; and Brij (trademark) 35, Brij (trademark) 58, Brij (trademark) 76, Brij (trademark) 98, Triton (trademark) X-100, Triton (trademark) X-114, Triton (trademark) X-305, Triton (trademark) N-101, Nonidet (trademark) P-40, IGEPAL (trademark) CO 530, IGEPAL (trademark) CO 630, IGEPAL (trademark) CO 720, and IGEPAL (trademark) CO 730.

In addition, examples of the anionic surfactant can include sodium dodecyl sulfate, dodecyl benzenesulfonate, decyl benzenesulfonate, undecyl benzenesulfonate, tridecyl benzenesulfonate, and nonyl benzenesulfonate, and sodium, potassium, and ammonium salts thereof.

In addition, examples of the cationic surfactant can include cetyltrimethylammonium bromide, hexadecylpyridinium chloride, dodecyltrimethylammonium chloride, and hexadecyltrimethylammonium chloride.

In addition, examples of the polymeric surfactant can include polyvinyl alcohol and polyoxyethylene polyoxypropylene glycol. As a commercial product of the polyoxyethylene polyoxypropylene glycol, there can be given, for example, Pluronic F68 (manufactured by BASF) and Pluronic F127 (manufactured by BASF).

Examples of the phospholipid can include: a phosphatidyl phospholipid having any one of an amino group, an NHS group, a maleimide group, or methoxy group as a functional group, and a PEG chain; and a phospholipid having a phosphodiester bond.

Examples of the phosphatidyl phospholipid include 3-(N-succinimidyloxyglutaryl)aminopropyl, polyethyleneglycol-carbamyl distearoylphosphatidyl-ethanolamine (DSPE-PEG-NHS), N-(3-maleimide-1-oxopropyl)aminopropyl polyethyleneglycol-carbamyl distearoylphosphatidyl-ethanolamine (DSPE-PEG-MAL), N-(aminopropyl polyethyleneglycol)-carbamyl distearoylphosphatidyl-ethanolamine (DSPE-PEG-NH2), N-(carbonyl-methoxypolyethyleneglycol 2000)-1,2-distearoyl-sn-glycero-3-phosphoethanolamine, sodium salt (SUNBRIGHT DSPE-020CN), and N-(carbonyl-methoxypolyethyleneglycol 5000)-1,2-distearoyl-sn-glycero-3-phosphoethanolamine, sodium salt (SUNBRIGHT DSPE-050CN).

Examples of the phospholipid having a phosphodiester bond can include 1,2-distearoyl-sn-glycero-3-phosphoethanolamine (DSPE), 1,2-dipalmitoyl-sn-glycero-3-phosphoethanolamine (DPPE), 1,2-dimyristoyl-sn-glycero-3-phosphoethanolamine (DMPE), 1,2-dilauroyl-sn-glycero-3-phosphoethanolamine (DLPE), 1,2-dioleoyl-sn-glycero-3-phosphoethanolamine (DOPE), 1,2-dilinoleoyl-sn-glycero-3-phosphoethanolamine (DLoPE), 1,2-dierucoyl-sn-glycero-3-phosphoethanolamine (DEPE), 1,2-distearoyl-snglycero-3-phospho-L-serine (DSPS), 1,2-dipalmitoyl-sn-glycero-3-phospho-L-serine (DPPS), 1,2-dimyristoyl-sn-glycero-3-phospho-L-serine (DMPS), 1,2-dioleoyl-sn-glycero-3-phospho-L-serine (DOPS), 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dilauroyl-sn-glycero-3-phosphocholine (DLPC), 1,2-dioleoyl-sn-glycero-3-phosphocholine (DOPC), and 1,2-dilinoleoyl-sn-glycero-3-phosphocholine (DLoPC).

(Matrix Material)

Although any material may be used as the matrix material as long as the material can include the hydrophobic metal naphthalocyanine dye or the hydrophobic metal phthalocyanine dye, a material having a large coefficient of thermal expansion and a small specific heat capacity is preferably used in consideration of an amplifying effect on a photoacoustic signal.

Examples of the matrix material in this embodiment can include an oil, a fatty acid, and a fatty acid ester.

Examples of the oil include salad oil, olive oil, safflower oil, canola oil, sesame oil, corn oil, Japanese basil oil, soybean oil, safflower oil, rapeseed oil, palm oil, cottonseed oil, perilla oil, peanut oil, flavor oil, sunflower oil, coconut oil, rice oil (rice bran oil), and maize oil.

In addition, examples of the fatty acid include palmitic acid, stearic acid, oleic acid, linoleic acid, and linolenic acid.

In addition, examples of the fatty acid ester include: a triglyceride having a palmitic acid, stearic acid, oleic acid, linoleic acid, or linolenic acid structure; an alkyl ester having a palmitic acid, stearic acid, oleic acid, linoleic acid, or linolenic acid structure; and cholesterol having an ester bond with palmitic acid, stearic acid, oleic acid, linoleic acid, or linolenic acid.

When the particle contains a large amount of the matrix material, the dye hardly leaks to the outside of the particle even in an aqueous solution such as serum. Accordingly, the weight ratio of the matrix material with respect to the entirety of the particle is preferably 50% or more.

(Method of Producing Particle)

A known method can be utilized as a method of producing the particle of the present invention. Examples thereof can include a nanoemulsion method and a nanoprecipitation method.

As a solvent to be used in this production method, there can be given, for example: a hydrocarbon such as hexane, cyclohexane, or heptane; a ketone such as acetone or methyl ethyl ketone; an ether such as diethyl ether or tetrahydrofuran; a halogenated hydrocarbon such as dichloromethane, chloroform, carbon tetrachloride, dichloroethane, or trichloroethane; an aromatic hydrocarbon such as benzene or toluene; an ester such as ethyl acetate or butyl acetate; a non-protonic polar solvent such as N,N-dimethylformamide or dimethyl sulfoxide; and a pyridine derivative. One kind of those solvents may be used alone, or two or more kinds thereof may be arbitrarily mixed before use.

In the nanoemulsion method, an emulsion can be prepared by a conventionally known emulsification approach. Examples of the conventionally known method include an intermittent shaking method, a stirring method involving utilizing a mixer such as a propeller type stirring machine or a turbine type stirring machine, a colloid mill method, a homogenizer method, and an ultrasonic irradiation method. One kind of those methods can be employed alone, or two or more kinds thereof can be employed in combination. In addition, the emulsion may be prepared by one-stage emulsification, or may be prepared by multistage emulsification. It should be noted that the emulsification approach is not limited to the foregoing approaches to the extent that the object of the present invention can be achieved.

In the nanoprecipitation method, the particle can be prepared by a conventionally known method involving mixing a surfactant-dispersed aqueous solution with an organic solvent dispersion liquid and stirring the mixture, or a conventionally known method involving mixing the organic solvent dispersion liquid with the surfactant-dispersed aqueous solution and stirring the mixture.

In addition, in the production method, when the matrix material in which the hydrophobic metal naphthalocyanine dye or the hydrophobic metal phthalocyanine dye is soluble is used, a dye-dissolved matrix material solution and the surfactant can be mixed. At that time, the particle can be prepared by a conventionally known method involving mixing a mixed solution of the dye-dissolved matrix material solution and the surfactant with a dispersion medium, and stirring the mixture, or a conventionally known method involving mixing the dispersion medium with the mixed solution of the dye-dissolved matrix material solution and the surfactant, and stirring the mixture.

(Organic Solvent Dispersion Liquid Having Dissolved Therein Material Containing Hydrophobic Metal Naphthalocyanine Dye or Hydrophobic Metal Phthalocyanine Dye)

A weight ratio between the surfactant-dispersed aqueous solution and organic solvent dispersion liquid to be used in the nanoemulsion method is not particularly limited as long as an oil-in-water (O/W) type emulsion can be formed. The weight ratio between the organic solvent dispersion liquid and the aqueous solution preferably falls within the range of from 1:2 to 1:1,000.

A weight ratio between the surfactant-dispersed aqueous solution and organic solvent dispersion liquid to be used in the nanoprecipitation method is not particularly limited as long as the particle can be recovered. The weight ratio between the organic solvent dispersion liquid and the aqueous solution preferably falls within the range of from 1:1 to 1:1,000.

(Material Concentration in Organic Solvent Dispersion Liquid Having Dissolved Therein Material Containing Hydrophobic Metal Naphthalocyanine Dye or Hydrophobic Metal Phthalocyanine Dye)

The concentration of the hydrophobic metal naphthalocyanine dye or hydrophobic metal phthalocyanine dye in the organic solvent dispersion liquid is not particularly limited as long as the concentration falls within such a range that the dye dissolves therein. A preferred concentration can be from 0.0005 to 300 mg/ml.

The concentration of the matrix material in the organic solvent dispersion liquid is not particularly limited as long as the concentration falls within such a range that the material dissolves therein. A preferred concentration can be from 0.3 to 100 mg/ml. In addition, a weight ratio between the hydrophobic metal naphthalocyanine dye or hydrophobic metal phthalocyanine dye and matrix material in the organic solvent dispersion liquid preferably falls within the range of from 100:1 to 1:1,000.

(Distillation of Organic Solvent from Particle Dispersion Liquid)

Distillation can be performed by any one of the conventionally known methods, and examples thereof can include a method involving removal through heating and a method involving utilizing a pressure-reducing apparatus such as an evaporator.

In the nanoemulsion method, a heating temperature in the case of the removal through heating is not particularly limited as long as the O/W type emulsion can be maintained, but a preferred temperature falls within the range of from 0° C. to 80° C.

In the nanoprecipitation method, a heating temperature in the case of the removal through heating is not particularly limited as long as higher-order aggregation reducing the yield of the particle can be prevented, but a preferred temperature falls within the range of from 0° C. to 80° C.

It should be noted that the approach to the distillation is not limited to those described above to the extent that the object of the present invention can be achieved.

(Purification of Particle Dispersion Liquid)

The purification of the produced particle dispersion liquid can be performed by any one of the conventionally known methods. Examples thereof can include a size exclusion column chromatography method, an ultrafiltration method, a dialysis method, and a centrifugation method.

It should be noted that a method for the purification is not limited to the foregoing approaches to the extent that the object of the present invention can be achieved.

(Particle)

The particle according to this embodiment may be of any shape as long as the particle includes the hydrophobic metal naphthalocyanine dye or the hydrophobic metal phthalocyanine dye, and any one of the shapes such as a true spherical shape, an elliptical shape, a flat surface shape, and a one-dimensional string shape is permitted. The size (particle diameter) of the particle according to this embodiment, which is not particularly limited, is preferably 1 nm or more and 200 nm or less.

(Contrast Agent)

A contrast agent according to this embodiment includes the particle according to this embodiment and a dispersion medium. The dispersion medium is a liquid substance, and examples thereof include physiological saline, distilled water for injection, and phosphate buffered saline (hereinafter sometimes abbreviated as “PBS”). In addition, the contrast agent according to this embodiment may include a pharmacologically acceptable additive as required.

In the contrast agent according to this embodiment, the particle may be dispersed in the dispersion medium in advance, or the following may be adopted: the particle according to this embodiment and the dispersion medium are turned into a kit, and then the particle is dispersed in the dispersion medium and used before administration into a living organism.

The particle according to this embodiment contains a large amount of the hydrophobic metal naphthalocyanine dye or the hydrophobic metal phthalocyanine dye in itself because the hydrophobic metal naphthalocyanine dye or the hydrophobic metal phthalocyanine dye hardly leaks to the outside of the particle. As described later, the particle according to this embodiment is suitable for a photoacoustic imaging application because the quantity of light to be absorbed increases as the amount of the dye in the particle increases.

(Additive)

The contrast agent according to this embodiment may contain an additive to be used at the time of freeze-drying. Examples of the additive include glucose, lactose, mannitol, polyethylene glycol, glycine, sodium chloride, and disodium hydrogen phosphate. Only one kind of the additives may be used, or two or more kinds thereof may be used in combination.

(Photoacoustic Imaging Method)

The contrast agent according to this embodiment can be used in a photoacoustic imaging method. It should be noted that in the specification, photoacoustic imaging is a concept comprehending photoacoustic tomography. The photoacoustic imaging method involving using the contrast agent according to this embodiment includes at least the steps of: administering the contrast agent according to this embodiment to a specimen or a sample obtained from the specimen; irradiating the specimen or the sample obtained from the specimen with pulse light; and measuring the photoacoustic signal of a substance derived from the particle present in the specimen or in the sample obtained from the specimen.

An example of the photoacoustic imaging method involving using the contrast agent according to this embodiment is as described below. That is, the contrast agent according to this embodiment is administered to a specimen or is added to a sample such as an organ obtained from the specimen. It should be noted that the specimen is not particularly limited to a human being, an experimental animal, a pet, and other specimens, and refers to all kinds of living organisms, and examples of the sample present in the specimen or obtained from the specimen can include an organ, a tissue, a tissue section, a cell, and a cell lysate. After the administration or addition of the particle, the specimen or the like is irradiated with laser pulse light in a near-infrared wavelength region.

The wavelength of the light with which the specimen or the like is to be irradiated in the photoacoustic imaging method according to this embodiment can be selected depending on a laser light source to be used. In the photoacoustic imaging method according to this embodiment, in order that an acoustic signal may be efficiently acquired, the specimen or the like is preferably irradiated with light having a wavelength in a near-infrared light region ranging from 600 nm to 1,300 nm referred to as “biological window” where an influence of the absorption or diffusion of the light in a living organism is small.

A photoacoustic signal (acoustic wave) from the contrast agent according to this embodiment is detected with an acoustic wave detector such as a piezoelectric transducer and converted into an electric signal. The position or size of an absorber in the specimen or the like, or the optical characteristic value distribution of a molar absorption coefficient or the like can be calculated based on the electric signal obtained with the acoustic wave detector. For example, when the contrast agent is detected at a value equal to or more than a threshold as a reference, it can be assumed that the substance derived from the particle is present in the specimen or that the substance derived from the particle is present in the sample obtained from the specimen.

In the present invention, quenching based on the accumulation of the dye is caused by suppressing the leakage of the dye, and hence the energy of the pulse light with which the specimen or the like has been irradiated is prevented from being used in fluorescent emission and can be converted into an additionally large quantity of thermal energy. Accordingly, an acoustic signal can be acquired in an additionally efficient manner.

In addition, a particle in which a signal strength per unit dye is high because of the thermal expansion effect of the matrix material can be provided.

Hereinafter, the present invention is described by way of examples in order that the features of the present invention may be additionally clarified, but the present invention is not limited to these examples, and a material, a composition condition, a reaction condition, and the like can be freely changed to the extent that a contrast agent having the same function and effect is obtained.

(Analysis Method)

Particle diameter measurement was performed with a dynamic light scattering analyzer (ELSZ-2 manufactured by Otsuka Electronics Co., Ltd.).

The measurement was performed by using a semiconductor laser as a light source and a value for a cumulant diameter was adopted as a particle diameter.

The concentration of a surfactant in a sample was calculated by NMR measurement (AVANCE 500 manufactured by Bruker; resonance frequency: 500 MHz; measurement nuclear species: 1H; measurement temperature: room temperature; solvent: heavy chloroform).

In addition, the concentration of a hydrophobic metal naphthalocyanine dye or hydrophobic metal phthalocyanine dye in the sample was calculated by absorbance measurement (Lambda Bio 40 manufactured by PerkinElmer, Inc.).

After the sample had been freeze-dried, its solid weight was calculated, and the weight of a matrix was calculated by subtracting the weight of the surfactant and the concentration by weight of the hydrophobic metal naphthalocyanine dye or the hydrophobic metal phthalocyanine dye from the solid weight.

(Method for Evaluation for Photoacoustic Characteristic)

Photoacoustic signal measurement was performed as described below. The sample was irradiated with pulse laser light, a photoacoustic signal from the sample was detected with a piezoelectric element, the signal was amplified with a high-speed preamplifier, and the amplified signal was acquired with a digital oscilloscope. Specific conditions for the measurement are as described below. A titanium sapphire laser (manufactured by Lotis Ltd.) was used as a light source. The conditions of a wavelength of 750 nm or 780 nm, an energy density of 12 mJ/cm², a pulse width of 20 nanoseconds, and a pulse repetition of 10 Hz were adopted. A Model V303 (manufactured by Panametrics-NDT) was used as an ultrasonic transducer. The conditions of a central band of 1 MHz, an element size of φ0.5, a measurement distance of 25 mm (non-focus), and an amplification of +30 dB (Ultrasonic Preamplifier Model 5682 manufactured by Olympus Corporation) were adopted. A measurement vessel was a cuvette made of polystyrene, and had an optical path length of 0.1 cm and a sample volume of about 200 μl. A DPO4104 (manufactured by TEKTRONIX, INC.) was used as a measuring device, and measurement was performed under the conditions of: trigger: detection of photoacoustic light with a photodiode; and data acquisition: 128 times (128 pulses) on average.

(Method of Evaluating Hydrophobic Metal Naphthalocyanine Dye or Hydrophobic Metal Phthalocyanine Dye for its Hydrophobicity)

An evaluation was performed by employing a thin-layer liquid chromatography (hereinafter sometimes abbreviated as “TLC”) method for the comparison of the hydrophobicity of the hydrophobic metal phthalocyanine dye.

A TLC glass plate RP-18 (manufactured by Merck) was used as a plate for development and a methanol solution containing 1 wt % of lithium chloride was used as a developing solvent.

A dye solution was spotted on an initial line according to an ordinary method and a relative movement distance (hereinafter sometimes abbreviated as “Rf value”) was calculated based on the following equation.

Rf value=distance from initial line to spot center of component/distance from initial line to solvent front.

### Example A1

2.0 Milliliters of oleic acid (manufactured by Tokyo Chemical Industry Co., Ltd.) were added to 10 mg of silicon 2,3-naphthalocyanine bis(trihexylsilyloxide)<Rf value=0> (manufactured by Sigma-Aldrich) as a dye.

After having been stirred for 15 minutes under heating at 70° C., the mixture was filtered with a filter having a pore diameter of 0.20 μm to prepare a dye-dissolved matrix material solution.

60 Milligrams of Tween 80 (manufactured by NOF CORPORATION) were added as a surfactant to the solution and the mixture was stirred for 15 minutes. While the mixture was stirred, 13.0 mL of ultrapure water were added to the mixture. Thus, a particle dispersion liquid was produced.

The recovered particle dispersion liquid was filtered with a filter having a pore diameter of 1.20 μm to provide a particle (A-1).

### Example A2

A particle (A-2) was produced in the same manner as in Example A1 except that the usage of Tween 80 (manufactured by NOF CORPORATION) was changed to 12 mg.

### Comparative Example B1

A particle (B-1) was obtained by producing a dye-solubilized solution according to Investigative Ophthalmology & Visual Science, November 1995, Vol. 36, No. 12 except that canola oil was changed to oleic acid.

### Example C1

2.5 Milliliters of canola oil (manufactured by Sigma-Aldrich) were added to 5.0 mg of silicon 2,3-naphthalocyanine bis(trihexylsilyloxide) (manufactured by Sigma-Aldrich) as a dye.

After having been stirred for 15 minutes under heating at 70° C., the mixture was filtered with a filter having a pore diameter of 0.20 μm to prepare a dye-dissolved matrix material solution.

75 Milligrams of Tween 80 (manufactured by NOF CORPORATION) were added as a surfactant to 88.0 mg of the dye-dissolved matrix material solution and the mixture was stirred for 15 minutes. While the mixture was stirred, 13.0 mL of ultrapure water were added to the mixture. Thus, a particle dispersion liquid was produced.

The recovered particle dispersion liquid was filtered with a filter having a pore diameter of 1.20 μm to provide a particle (C-1).

### Comparative Example D1

A particle (D-1) was obtained by producing a dye-solubilized solution according to Investigative Ophthalmology & Visual Science, November 1995, Vol. 36, No. 12.

### Example E1

2.0 Milliliters of oleic acid (manufactured by Tokyo Chemical Industry Co., Ltd.) were added to 10 mg of zinc 2,9,16,23-tetra-tert-butyl-29H,31H-phthalocyanine <Rf value=0.10> as a dye.

After having been stirred for 15 minutes under heating at 70° C., the mixture was filtered with a filter having a pore diameter of 0.20 μm to prepare a dye-dissolved matrix material solution.

60 Milligrams of Tween 20 (manufactured by NOF CORPORATION) were added as a surfactant to the solution and the mixture was stirred for 15 minutes. While the mixture was stirred, 13.0 mL of ultrapure water were added to the mixture. Thus, a particle dispersion liquid was produced.

The recovered particle dispersion liquid was filtered with a filter having a pore diameter of 1.20 μm to provide a particle (E-1).

### Example E2

A particle (E-2) was produced in the same manner as in Example C-1 except that Tween 80 was used as a surfactant.

### Comparative Example F1

8.8 Milligrams of zinc 2,9,16,23-tetra-tert-butyl-29H,31H-phthalocyanine as a dye were dissolved in 16.0 mL of chloroform.

1,800 Milligrams of Tween 20 (manufactured by Wako Pure Chemical Industries, Ltd.) were added as a surfactant to 200 mL of ultrapure water to prepare a surfactant-dispersed aqueous solution

While the surfactant-dispersed aqueous solution was stirred, the organic solvent dispersion liquid was dropped. Thus, an emulsion preparation liquid was prepared.

The emulsion preparation liquid was irradiated with an ultrasonic wave for 1 minute and 30 seconds by using an ultrasonic wave disperser (UD-200 manufactured by TOMY SEIKO CO., LTD.) with its strength scale set to 10. Thus, an emulsion was produced.

In order for chloroform in the emulsion to be removed, the emulsion was stirred for 4 hours under heating at 40° C. Thus, a particle dispersion liquid was produced.

The recovered particle dispersion liquid was filtered with a filter having a pore diameter of 0.20 μm to provide a particle (F-1).

### Comparative Example F2

11.5 Milligrams of zinc 2,9,16,23-tetra-tert-butyl-29H,31H-phthalocyanine as a dye were dissolved in 20.9 mL of chloroform to prepare a dye solution.

0.8 Milliliter of the dye solution was diluted with 0.8 mL of chloroform to produce a solution having a dye concentration of 0.28 mg/mL. The solution was used in the following operation.

5 Milligrams of PLGA (manufactured by Wako Pure Chemical Industries, Ltd.) were added as a matrix material to the solution to prepare an organic solvent dispersion liquid.

180 Milligrams of Tween 20 (manufactured by Wako Pure Chemical Industries, Ltd.) were added as a surfactant to 20 mL of ultrapure water to prepare a surfactant-dispersed aqueous solution.

While the surfactant-dispersed aqueous solution was stirred, the organic solvent dispersion liquid was dropped. Thus, an emulsion preparation liquid was prepared.

The emulsion preparation liquid was irradiated with an ultrasonic wave for 1 minute and 30 seconds by using an ultrasonic wave disperser (UD-200 manufactured by TOMY SEIKO CO., LTD.) with its strength scale set to 4. Thus, an emulsion was produced.

In order for chloroform in the emulsion to be removed, the emulsion was stirred for 2 hours under heating at 40° C. Thus, a particle dispersion liquid was produced.

The resultant particle dispersion liquid was recovered by being centrifuged at 4° C. and 20,000 G for 45 minutes.

The recovered particle was washed with ultrapure water. After that, the particle was recovered by being centrifuged at 4° C. and 20,000 G for 45 minutes.

The recovered particle dispersion liquid was filtered with a filter having a pore diameter of 0.20 μm to provide a particle (F-2).

### Example G1

0.88 Milligram of silicon 2,3-naphthalocyanine bis(trihexylsilyloxide) (manufactured by Sigma-Aldrich) as a dye was dissolved in 1.6 mL of chloroform.

10.0 Milligrams of canola oil (manufactured by Sigma-Aldrich) were added as a matrix material to the solution to prepare an organic solvent dispersion liquid.

6.9 Milligrams of 1,2-distearoyl-sn-glycero-3-phosphocholine (DSPC) (manufactured by NOF CORPORATION) were added as a surfactant to the organic solvent dispersion liquid.

3.2 Milligrams of Tween 80 (manufactured by NOF CORPORATION) were added as a surfactant to 20 mL of ultrapure water to prepare a surfactant-dispersed aqueous solution.

While the surfactant-dispersed aqueous solution was stirred, the organic solvent dispersion liquid was dropped. Thus, an emulsion preparation liquid was prepared.

The emulsion preparation liquid was irradiated with an ultrasonic wave for 1 minute and 30 seconds by using an ultrasonic wave disperser (UD-200 manufactured by TOMY SEIKO CO., LTD.) with its strength scale set to 10. Thus, an emulsion was produced.

In order for chloroform in the emulsion to be removed, the emulsion was stirred for 4 hours under heating at 40° C. Thus, a particle dispersion liquid was produced.

The recovered particle dispersion liquid was purified by dialysis involving using a dialysis membrane (manufactured by Spectrum Laboratories, Inc., cutoff molecular weight: 3,000,000) and ultrapure water as a solvent.

The purified liquid was filtered with a filter having a pore diameter of 1.20 μm to provide a particle (G-1).

### Example G2

A particle (G-2) was produced in the same manner as in Example G1 except that: the addition amount of ultrapure water was changed to 200 mL; and 20 mL of the surfactant-dispersed aqueous solution were used.

### Example G3

A particle (G-3) was produced in the same manner as in Example G1 except that: the addition amount of ultrapure water was changed to 2,000 mL; and 20 mL of the surfactant-dispersed aqueous solution were used.

### Comparative Example H1

A particle (H-1) was produced in the same manner as in Example G1 except that the used amount of Tween 80 (manufactured by NOF CORPORATION) was changed to 32.0 mg.

### Example J1

A particle (J-1) was produced in the same manner as in Example G1 except that: the used amount of Tween 80 (manufactured by NOF CORPORATION) was changed to 32.0 mg; and 10.0 mg of cholesterol oleate were used as a matrix material.

### Example J2

A particle (J-2) was produced in the same manner as in Example J1 except that the used amount of Tween 80 (manufactured by NOF CORPORATION) was changed to 3.2 mg.

### Example J3

A particle (J-3) was produced in the same manner as in Example J1 except that: the addition amount of ultrapure water was changed to 200 mL; and 20 mL of the surfactant-dispersed aqueous solution were used.

### Comparative Example K1

A particle (K-1) was produced in the same manner as in Example J1 except that no matrix material was used.

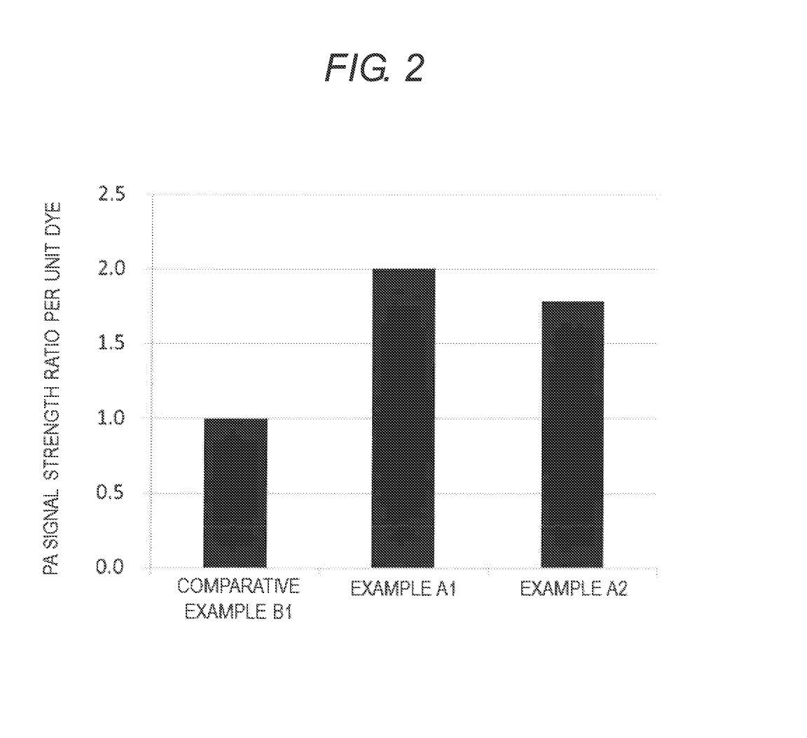

FIG. 2 shows a ratio among photoacoustic signal strengths per unit dye (at a wavelength of 780 nm) in the particles (A-1), (A-2), and (B-1) obtained in the foregoing.

Table 1 shows the weight ratios of the matrix materials in the particles (A-1), (A-2), and (B-1) obtained in the foregoing.

TABLE 1

| | Comparative | | |
|---|---|---|---|
| | Example B1 | Example A1 | Example A2 |
| Particle | B-1 | A-1 | A-2 |
| Weight ratio of | 46 | 59 | 88 |
| matrix material (%) | | | |

The particles (A-1) and (A-2) produced in Examples each had a higher weight ratio of the matrix material and a higher photoacoustic signal strength per unit dye (at a wavelength of 780 nm) than those of the particle (B-1) of Comparative Example.

Therefore, the particles according to Examples are each suitable as a contrast agent for photoacoustic imaging.

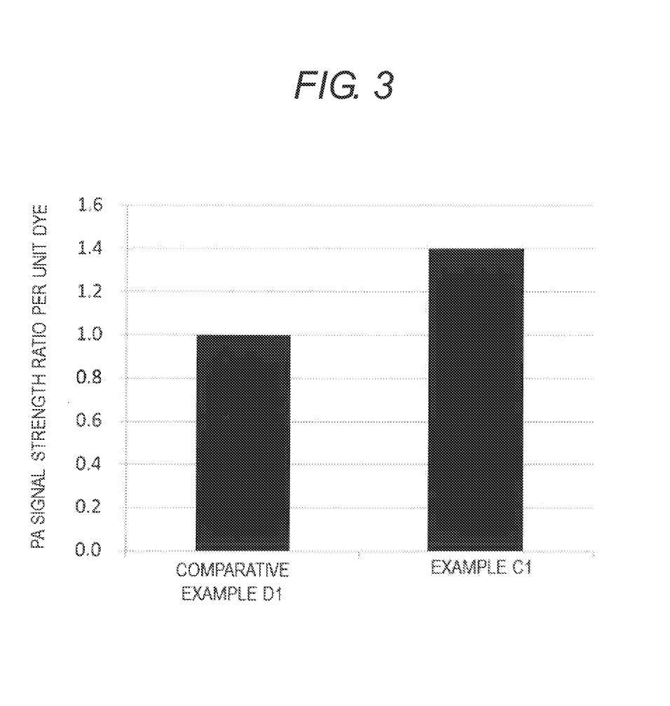

FIG. 3 shows a ratio between photoacoustic signal strengths per unit dye (at a wavelength of 780 nm) in the particles (C-1) and (D-1) obtained in the foregoing.

Table 2 shows the weight ratios of the matrix materials in the particles (C-1) and (D-1) obtained in the foregoing.

TABLE 2

| | Comparative | |
|---|---|---|
| | Example D1 | Example C1 |
| Particle | D-1 | C-1 |
| Weight ratio of | 45 | 54 |
| matrix material (%) | | |

The particle (C-1) produced in Example each had a higher weight ratio of the matrix material and a higher photoacoustic signal strength per unit dye (at a wavelength of 780 nm) than those of the particle (D-1) of Comparative Example.

Therefore, the particle according to Example is suitable as a contrast agent for photoacoustic imaging.

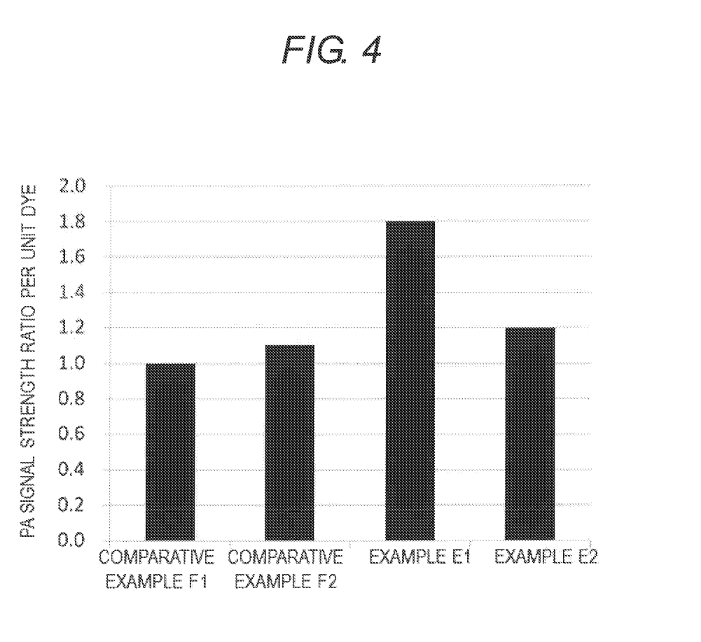

FIG. 4 shows a ratio among photoacoustic signal strengths per unit dye (at a wavelength of 750 nm) in the particles (E-1), (E-2), (F-1), and (F-2) obtained in the foregoing.

Table 3 shows the kinds and weight ratios of the matrix materials in the particles (E-1), (E-2), (F-1), and (F-2) obtained in the foregoing.

TABLE 3

| | Comparative | Comparative | | |
|---|---|---|---|---|
| | Example F1 | Example F2 | Example E1 | Example E2 |
| Particle | F-1 | F-2 | E-1 | E-2 |
| Kind of | None | PLGA | Oleic acid | Oleic acid |
| matrix | | | | |
| material | | | | |
| Weight | — | 81 | 88 | 84 |
| ratio of | | | | |
| matrix | | | | |
| material (%) | | | | |

The particles (E-1) and (E-2) produced in Examples each had a higher photoacoustic signal strength per unit dye (at a wavelength of 750 nm) than those of the particles (F-1) and (F-2) of Comparative Examples.

Therefore, the particles according to Examples are each suitable as a contrast agent for photoacoustic imaging.

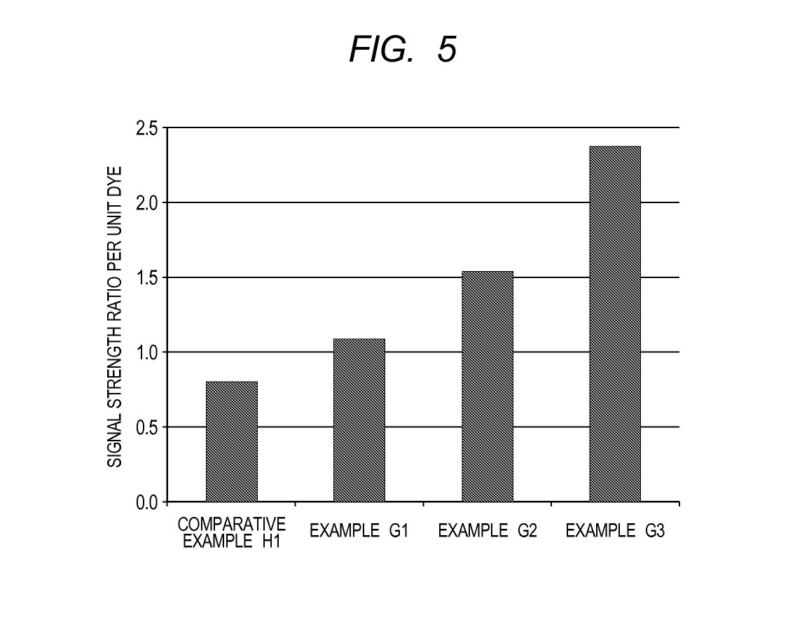

FIG. 5 shows a ratio among photoacoustic signal strengths per unit dye (at a wavelength of 780 nm) in the particles (G-1), (G-2), (G-3), and (H-1) obtained in the foregoing.

Table 4 shows the kinds and weight ratios of the matrix materials in the particles (G-1), (G-2), (G-3), and (H-1) obtained in the foregoing.

TABLE 4

| | Comparative | | | |
|---|---|---|---|---|
| | Example H1 | Example G1 | Example G2 | Example G3 |
| Particle | H-1 | G-1 | G-2 | G-3 |
| Weight | 45 | 64 | 78 | 78 |
| ratio of | | | | |
| matrix | | | | |
| material (%) | | | | |

The particles (G-1), (G-2), and (G-3) produced in Examples each had a higher photoacoustic signal strength per unit dye (at a wavelength of 780 nm) than that of the particle (H-1) of Comparative Example.

Therefore, the particles according to Examples are each suitable as a contrast agent for photoacoustic imaging.

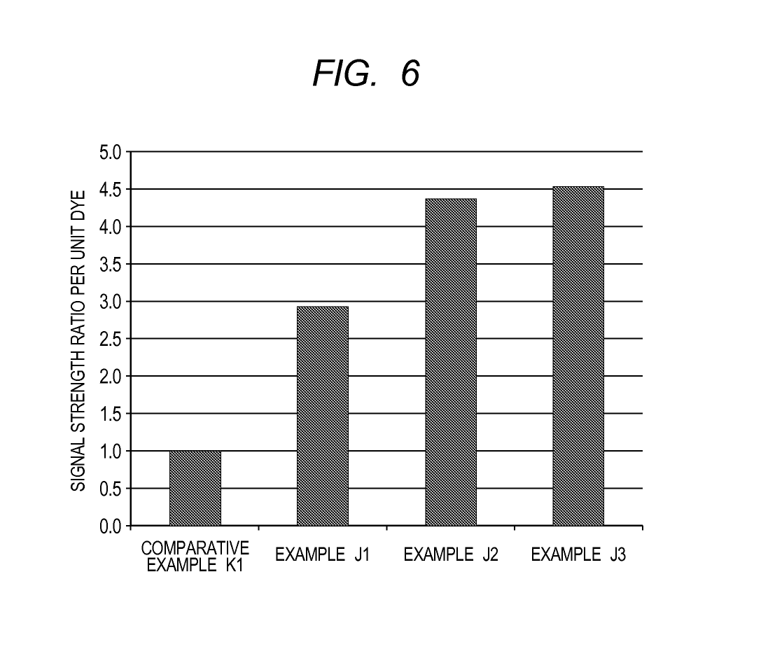

FIG. 6 shows a ratio among photoacoustic signal strengths per unit dye (at a wavelength of 780 nm) in the particles (J-1), (J-2), (J-3), and (K-1) obtained in the foregoing.

Table 5 shows the kinds and weight ratios of the matrix materials in the particles (J-1), (J-2), (J-3), and (K-1) obtained in the foregoing.

TABLE 5

| | Comparative | | | |
|---|---|---|---|---|
| | Example K1 | Example J1 | Example J2 | Example J3 |
| Particle | K-1 | J-1 | J-2 | J-3 |
| Weight | 0 | 57 | 63 | 66 |
| ratio of | | | | |
| matrix | | | | |
| material (%) | | | | |

The particles (J-1), (J-2), and (J-3) produced in Examples each had a higher photoacoustic signal strength per unit dye (at a wavelength of 780 nm) than that of the particle (K-1) of Comparative Example.

Therefore, the particles according to Examples are each suitable as a contrast agent for photoacoustic imaging.

(Confirmation of Tumor Accumulation Property)

In the confirmation of tumor accumulation property, female outbred BALB/c Slc-nu/nu mice (6-week old at the time of purchase) (Japan SLC, Inc.) were used. For 1 week before the causing of the mice to bear cancers, the mice were habituated with a normal diet and bed under such an environment that the diet and drinking water were available ad libitum. Colon 26 (mouse colon cancer cell) was subcutaneously injected into the mice. All tumors fixed by the time an experiment was performed, and the mice each had a body weight of 17 to 22 g. 100 Microliters (13 nmol in terms of the dye) of a dispersion liquid of the particle (G-2) were intravenously injected into the mouse tail of each mouse caused to bear a cancer.

Next, the mouse having administered thereto the particle dispersion liquid was euthanized 24 hours after the administration, followed by the extirpation of the colon 26 tumor. The tumor tissue was transferred to a plastic tube and then homogenized by adding a 1% Triton-X100 aqueous solution in an amount 1.25 times as large as the weight of the tumor tissue thereto. Next, tetrahydrofuran (THF) was added in an amount 20.25 times as large as the weight of the tumor tissue thereto. The amount of the dye in the tumor tissue was determined by measuring the fluorescence intensity of the homogenized solution with an Odyssey (trademark) CLx Infrared Imaging System.

As a result, the tumor accumulation amount of the particle (G-2) was found to be 8% ID/g.

Therefore, the particle according to Example is suitable as a contrast agent for photoacoustic imaging.

The present invention can provide the following particle: the particle includes a matrix material capable of amplifying a photoacoustic signal and hence a photoacoustic signal per unit dye is large when the particle is used as a contrast agent for photoacoustic imaging.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-158053, filed Jul. 30, 2013, which is hereby incorporated by reference herein in its entirety.

What is claimed is:

1. A particle comprising:
one of a hydrophobic metal naphthalocyanine dye and a hydrophobic metal phthalocyanine dye;
one of an oil, a fatty acid, and a fatty acid ester as a matrix material; and
a surfactant,
wherein a ratio of a weight of the matrix material to a weight of the particle is 50% or more.

2. The particle according to claim 1, wherein one of the hydrophobic metal naphthalocyanine dye and the hydrophobic metal phthalocyanine dye is represented by one of the chemical formulae (1) and (2): 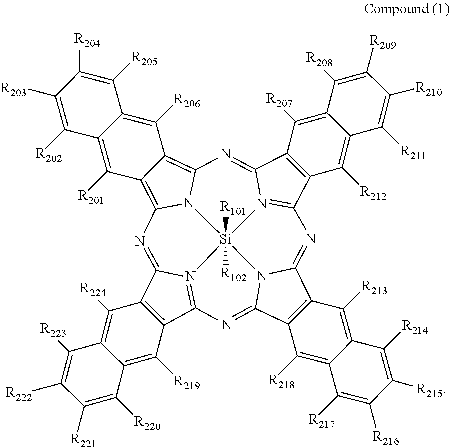
in the formula:
R₂₀₁ to R₂₂₄ may be identical to or different from one another, and each represent a hydrogen atom, a halogen atom, an acetoxy group, an amino group, a nitro group, a cyano group, or an alkyl group having 1 to 18 carbon atoms or an aromatic group that is unsubstituted or substituted with at least one functional group selected from a halogen atom, an acetoxy group, an amino group, a nitro group, a cyano group, and an alkyl group having 1 to 18 carbon atoms;
R₁₀₁ and R₁₀₂ may be identical to or different from each other, and each represent —OH, —OR₁₁, —OCOR₁₂, —OSi(—R₁₃)(—R₁₄)(—R₁₅), a halogen atom, an acetoxy group, an amino group, a nitro group, a cyano group, or an alkyl group having 1 to 18 carbon atoms or an aromatic group that is unsubstituted or substituted with at least one functional group selected from a halogen atom, an acetoxy group, an amino group, a nitro group, a cyano group, and an alkyl group having 1 to 18 carbon atoms; and
R₁₁, R₁₂, R₁₃, R₁₄, and R₁₅ may be identical to or different from one another, and each represent a group that is unsubstituted or substituted with at least one functional group selected from a halogen atom, an acetoxy group, an amino group, a nitro group, a cyano group, and an alkyl group having 1 to 18 carbon atoms; 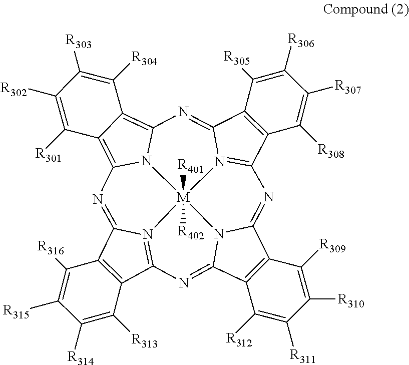
in the formula:
R₃₀₁ to R₃₁₆ may be identical to or different from one another, and each represent a hydrogen atom, a halogen atom, an acetoxy group, an amino group, a nitro group, a cyano group, or an alkyl group having 1 to 18 carbon atoms or an aromatic group that unsubstituted or is substituted with at least one functional group selected from a halogen atom, an acetoxy group, an amino group, a nitro group, a cyano group, and an alkyl group having 1 to 18 carbon atoms;
M represents one of elements Zn, Cu, Co, and Si;
R₄₀₁ and R₄₀₂ may be absent depending on the element represented by M, or each represent a structure described below;
R₄₀₁ and R₄₀₂ may be identical to or different from each other, and each represent —OH, —OR₅₀₁, —OCOR₅₀₂, —OSi(—R₅₀₃)(—R₅₀₄)(—R₅₀₅), a halogen atom, an acetoxy group, an amino group, a nitro group, a cyano group, or an alkyl group having 1 to 18 carbon atoms or an aromatic group that is unsubstituted or substituted with at least one functional group selected from a halogen atom, an acetoxy group, an amino group, a nitro group, a cyano group, and an alkyl group having 1 to 18 carbon atoms; and
R₅₀₁, R₅₀₂, R₅₀₃, R₅₀₄, and R₅₀₅ may be identical to or different from one another, and each represent a group that is unsubstituted or substituted with at least one functional group selected from a halogen atom, an acetoxy group, an amino group, a nitro group, a cyano group, and an alkyl group having 1 to 18 carbon atoms.

3. The particle according to claim 1, wherein one of the hydrophobic metal naphthalocyanine dye and the hydrophobic metal phthalocyanine dye comprises one of silicon 2,3-naphthalocyanine bis(trihexylsilyloxide) and zinc 2,9,16,23-tetra-tert-butyl-29H,31H-phthalocyanine.

4. The particle according to claim 1, wherein the matrix material comprises one of oleic acid, canola oil, and cholesterol oleate.

5. The particle according to claim 1, wherein the surfactant comprises a nonionic surfactant.

6. The particle according to claim 1, wherein the surfactant comprises a polyoxyethylene sorbitan-based fatty acid ester.

7. The particle according to claim 1, wherein the surfactant comprises one of Tween 20 and Tween 80.

8. A particle comprising:
one of a hydrophobic metal naphthalocyanine dye and a hydrophobic metal phthalocyanine dye;
a molecule having a higher coefficient of thermal expansion than that of water as a matrix material; and
a surfactant,
wherein a ratio of a weight of the matrix material to a weight of the particle is 50% or more.

9. A particle comprising:
one of a hydrophobic metal naphthalocyanine dye and a hydrophobic metal phthalocyanine dye;
a molecule having a lower specific heat capacity than that of water as a matrix material; and
a surfactant,
wherein a ratio of a weight of the matrix material to a weight of the particle is 50% or more.

10. A contrast agent for photoacoustic imaging, comprising:
the particle according to claim 1; and
a dispersion medium.

11. The contrast agent according to claim 10, further comprising an additive.
